(19) United States
(12) Patent Application Publication
(10) Pub. No.: US 2017/0223976 A1
(43) Pub. Date: Aug. 10, 2017

(54) MODIFIED MILK POWDER CONTAINING 1,5-ANHYDRO-D-GLUCITOL

(71) Applicant: NIHON STARCH CO., LTD., Kagoshima (JP)

(72) Inventor: Kazuhiro YOSHINAGA, Kagoshima (JP)

(73) Assignee: NIHON STARCH CO., LTD., Kagoshima (JP)

(21) Appl. No.: 14/912,653

(22) PCT Filed: May 8, 2015
(86) PCT No.: PCT/JP2015/063960

§ 371 Date: Feb. 18, 2016

(51) Int. Cl. A23C 9/152; C07H 3/10; A23L 33/00

(52) U.S. Cl. CPC A23C 9/152; A23L 33/40; C07H 3/10; A23V 2002/00

(57) ABSTRACT

A modified milk powder containing 1,5-anhydro-D-glucitol which is an effective component in vivo in an amount of 7.7 to 3,850 μg/g.

### TECHNICAL FIELD

The present invention provides a modified milk powder which contains a nutrient component able to be added to a modified milk powder for babies, pregnant women and nursing mothers and has the function of promoting neuritogenesis, the function of activating the expression of a pregnancy specific glycoprotein-1 (PSG-1) gene and the function of activating the expression of a chorionic somatomammotropin hormones 1 (CSH-1) gene.

### BACKGROUND ART

It is considered that 1,5-anhydro-D-glucitol (1,5-AG) is synthesized from glycogen through 1,5-anhydro-D-fructose (1,5-AF) as an intermediate in mammals, and 1,5-AG is contained in the blood of a normal human in a concentration amount of about 10 to 20 μg/mL. It is found that the 1,5-AG level in the blood is maintained constant in a healthy human. When the glucose level in the blood becomes high due to diabetes, the 1,5-AG level in the blood lowers correlatively, and the 1,5-AG level in the blood reflects the last transition of the blood sugar level. Therefore, the 1,5-AG level in the blood is used as an index for blood sugar control in a clinical stage.

Further, it is reported that 1,5-AG can be synthesized by the microbial conversion of 1,5-AF (refer to JP-A 2009-215231) and the chemical hydrogenation of 1,5-AF (refer to 1,5-anhydro-D-fructose; a versatile chiral building block: biochemistry and chemistry, Carbohydrate Research 337 (2002) 873-890). As a method of preparing high-purity 1,5-AG, a crystallization method is proposed (refer to JP-A 2008-54531). It is found that 1,5-AG promotes the secretion of insulin in a test system using the cell of pancreas as physiology (refer to 1,5-anhydroglucitol stimulates insulin release in insulinoma cell lines. Biochimica Biophysica Acta 1623 (2003) p. 82-87) and that 1,5-AG has an anti-inflammatory effect in a test system using type 2 diabetes model mice and may possibly be effective sugar for diabetic patients (refer to 1,5-anhydroglucitol attenuates cytokine releases and protect mice with type 2 diabetes from inflammatory reactions. Int. J. Immunopathol Pharmacol. 23 (2010) 105-119). Further, it is also reported that 1,5-AG has an anti-diabetic effect in a test using a type 2 diabetes model (refer to protective effects of dietary 1,5-anhydro-D-glucitol as a blood glucose regulator in diabetes and metabolic syndrome. J Argric Food Chem. 23 (2013) 611-7). Thus, 1,5-AG is a material which is expected to have healthy effects on humans.

1,5-AG is a component which is existent in mammals and various foods. It is reported that 0.6 μg of 1,5-AG is contained in 1 mL of bovine milk and 22 μg of 1,5-AG is contained in 1 g of soybeans (refer to Decomposition of Glycogen by Lyase and 1,5-anhydroglucitol, Biochemistry 69 (1997) 1361-1372).

The inventors of the present invention discovered that there is a big difference in the content of 1,5-AG between human breast milk and bovine milk when they determined the content of 1,5-AG in the several natural materials. That is, since it is reported that the content of 1,5-AG in breast milk is 2.6 to 12.3 μg/mL whereas the content of 1,5-AG in bovine milk is 0.6 μg/mL (refer to Decomposition of Glycogen by Lyase and 1,5-anhydroglucitol, Biochemistry 69 (1997) 1361-1372), it is found that 1,5-AG is contained in breast milk in an amount 10 times that of bovine milk. The contents of 1,5-AG in milk products prepared from commercially available modified milk powders for babies based on prescription (four products) were determined by the same operation as that for breast milk. As a result, the contents of 1,5-AG in these milk powders were not more than 1 μg/mL.

Although it is preferred that newborn babies should be breast-fed, there are quite a few mothers who use modified milk powders when the secretion of breast milk is bad or as the second best means for coping with a problem with the environment of child care. In Japan, it is said that babies who are fed only with modified milk powders account for 10%, babies who are fed with both breast milk and modified milk powders account for 60%, and babies who are fed only with breast milk account for 30%.

According to animal species, milk has component composition which differs between human and cow naturally. Modified milk powders are mainly produced from bovine milk. Since the components thereof differ from those of human milk, efforts have been made to make the composition of modified milk powders as close as possible to that of human milk and efforts to improve the composition of modified milk powders are still continued by modified milk powder manufactures.

Protein, fat and sugar are the main components of milk. There are big differences in protein and fat between human milk and bovine milk. The content of protein is 3.3% in bovine milk whereas the content of protein is 1.0 to 1.26% in human milk. Human milk and bovine milk also differ in the components of protein. As for casein, β-casein accounts for about 50 to 60% of human milk casein. κ-casein and α-casein are also contained. Meanwhile, bovine milk casein is composed of αs1-casein, αs2-casein, β-casein and κ-casein. Casein accounts for about 79% of all the proteins in bovine milk whereas casein accounts for about 12% in human milk.

Although 97 to 98% of both human milk fat and bovine milk fat is triacyl glycerol, there is a big difference in the composition of fatty acids constituting these fats, and these fats differ in the contents of oleic acid, linoleic acid and linolenic acid all of which are the main fatty acids. To reduce the differences in fatty acids, edible fat and oil may be added. Docosahexaenoic acid (DHA) is a component added as a raw material for modified milk powders, which softens the cytomembrane of a nerve cell, increases the number of dendrites, and promotes the growth of axons to keep brain and nerve systems healthy. Therefore, it is added as it is expected to provide these functions to modified milk powders. It is found that lactoferrin is contained in human milk and colostrum in particular is rich in lactoferrin.

Mothers tend to have poor nutrition during the pregnant period and the lactation period. As means for compensating for the deficiency of nutrition, powder milk for adults is also commercially available.

Although the significance of the existence of 1,5-AG in vivo is totally unknown, as for newborn babies who rely on milk as an only nutrient source, the intake of 1,5-AG by a newborn baby who is fed with breast milk is about 10 times that of a newborn baby who is fed with artificial milk. As for new “metabolic parameter 1-Deoxyglucose (1,5-Anhydroglucitol)” in diabetes, “Pediatric 24 (1983) 405-410” shows that the content of 1,5-AG in the blood of a 2-3 week-old baby is lower than that of a 2-3 month-old baby. When a baby is fed with breast milk, it can take a sufficient amount of 1,5-AG. However, a newborn baby who is fed with artificial milk falls short of 1,5-AG which is a component of breast milk. A fetus in mother's body grows while it is supplied nutrient components from mother through the placenta. When the content of 1,5-AG in mother's body, especially the blood decreases, the amount of 1,5-AG which the fetus can take also decreases.

The concentration of 1,5-AG in the blood is always maintained constant at 20 to 40 μg/mL in a normal human. If an excessive amount of 1,5-AG is taken as food, the concentration of 1,5-AG in the blood goes up to about 100 times temporarily but returns to the original level in about 24 hours. It is considered from this that the existence of 1,5-AG in a concentration of 20 to 40 μg/mL is meaningful.

### DISCLOSURE OF THE INVENTION

It is an object of the present invention to provide a modified milk powder containing 1,5-AG.

It is another object of the present invention to provide a modified milk powder containing 1,5-AG which serves to suppress retardation in neurogenesis, angiogenesis or intellectual development and enhancing of allergy, all of which may be caused by a reduction in the intake of 1,5-AG by babies.

It is still another object of the present invention to provide a modified milk powder containing 1,5-AG which has the strong effects of promoting neuritogenesis and activating the expression of PSG-1 gene and the expression of CSH-1 gene.

Other objects and advantages of the present invention will become obvious from the following description.

According to the present invention, the above objects and advantages of the present invention are attained by a modified milk powder which contains 1,5-anhydro-D-glucidol (1,5-AG) in an amount of 7.7 to 3,850 μg/g.

### BRIEF DESCRIPTION OF THE DRAWINGS

FIG. 1 shows 1,5-AG's function of expressing PSG-1 gene in a placental cell;

FIG. 2 shows 1,5-AG's function of expressing CSH-1 gene in a placental cell; and

FIG. 3 shows 1,5-AG's function of expressing PSG-1 gene in an intestinal tract cell.

### BEST MODE FOR CARRYING OUT THE INVENTION

According to studies conducted by the inventors of the present invention, as obvious from experiments in Examples 1 to 4 which will be described hereinafter, the reduction of the expression of PSG-1 gene and CSH-1 gene was observed due to the lack of 1,5-AG. When the content of 1,5-AG in the blood of an expectant mother decreases due to diabetes, the expression of PSG-1 or CSH-1 gene in the mother's body is suppressed with the result of the deterioration of the immuno-balance of the mother's body, a reduction in the supply of energy to a fetus, and the decrease of the amount of 1,5-AG to be supplied to a fetus from mother through the placenta, thereby reducing the amount of PSG-1 or CSH-1 gene of the fetus and retarding neurogenesis and angiogenesis. Therefore, there are concerns about a setback in the normal development of the fetus.

Since babies rely only on breast milk or modified milk powders for nutrition, when babies are fed only with a modified milk powder, a reduction in the intake of 1,5-AG causes retardation in neurological development and the reduction of the expression of PSG-1 gene, thereby inducing an abnormality in immuno-balance. Therefore, there are concerns about enhancing of allergy and retardation in intellectual development.

The modified milk powder of the present invention is effective in eliminating the above adverse effects and concerns and contains 1,5-AG in an amount of 7.7 to 3,850 μg/g, preferably 20 to 400 μg/g, more preferably 100 to 200 μg/g.

The upper limit value (3,850) in the above range is about 500 times larger than the lower limit value (7.7). A modified milk powder containing 1,5-AG in a lower limit amount of 7.7 μg/g may be used as a milk powder which is sometimes required for a baby which is fed only with breast milk, and a modified milk powder containing 1,5-AG in an upper limit amount of 3,850 μg/g suffices even when a baby is fed only with a milk powder. If the intake of 1,5-AG becomes large temporarily, it is readily discharged to the outside of the body, therefore causing no problem.

The modified milk powder is produced by mixing various additives with raw material milk through homogenizing, sterilizing, concentrating, drying and screening steps. As the method of adding 1,5-AG, a 1,5-AG solution or a 1,5-AG powder may be added to raw material milk. Also, a 1,5-AG powder may be mixed with a powder after drying. The method of adding 1,5-AG is not limited to these. 1,5-AG to be mixed into modified milk may be crystalline 1,5-AG or solution-like 1,5-AG, and the purity of 1,5-AG in the solid content of a 1,5-AG formulation is not less than 90% (W/W), preferably not less than 95%, much more preferably not less than 99%. When the purity of 1,5-AG is low, the risk of the coloration of contained impurities or the change of nutrient components becomes high. When a large amount of 1,5-anhydro-D-fructose (to be referred to as “1,5-AF” hereinafter) which is a raw material of 1,5-AG is contained as an impurity, a reaction with protein contained in the modified milk power is observed.

There are methods of producing 1,5-AG to be added, such as a method of chemically synthesizing 1,5-AG from glucose and a chemical hydrogenating method of 1,5-AF. Although the production method of 1,5-AG is not limited, the following method comprising no chemical production step is preferred. It is a method of producing 1,5-AG by fermenting microbiologically, refining, concentrating and crystallizing 1,5-AF, 1,5-AG produced by this method is suitable for food production. And 1,5-AG having higher purity is better.

### EXAMPLES

The following examples are provided to further illustrate the present invention.

### Example 1 (Measurement of the Content of 1,5-AG in Breast Milk)

Breast milk right after it was sampled about 1 week (colostrum) and about 1 month after delivery was offered from 10 women who gave birth at the department of obstetrics in Kagoshima city to measure the content of 1,5-AG in breast milk by using high-speed liquid chromatography and high-performance anion exchange chromatography. The measurement was made by the following method.

The sampled breast milk was put into a 1.5 mL centrifugal tube and kept in a −25° C. refrigerator until measurement. Before measurement, the breast milk was unfrozen. 300 μL of acetonitrile was added to and mixed with 300 μL of breast milk, and the obtained mixture was separated by centrifugation (10000×g) for 10 minutes to collect the supernatant. 100 μL of the supernatant was supplied into a high-speed liquid chromatograph (separation column; LiChroCARTLichrospher 100 NH₂, column temperature; 30° C., eluant; acetonitrile:water=70:30, flow rate; 1.0 mL/min) to collect a whole eluting 1,5-AG fraction of the solution eluting from the separation column. The collected fraction was supplied into a centrifugal evaporator, dried at 80° C. and dissolved in 100 μL of water. This sample was supplied into a high-performance anion exchange chromatogram (Dionex ICS5000+DC, separation column; Carbopac MA1, eluant; 400 mM NaOH, flow rate; 0.4 mL/min) to measure the concentration of 1,5-AG.

As a result, as shown in Table 1, it was found that 1,5-AG was contained in human breast milk. The concentration of 1,5-AG was 2.6 to 12.3 μg/mL and 6.0 μg/mL on average.

TABLE 1
Content of 1,5-AG in breast milk

| | |
|---|---|
| Number of human | Content of 1,5-AG (μg/mL) |

| breast milk | | Breast milk 1 month |
|---|---|---|
| suppliers | Colostrum | after delivery |
| 1 | 2.6 | 5.7 |
| 2 | 7.5 | 10.1 |
| 3 | 9.2 | 12.3 |
| 4 | 5.1 | 8.2 |
| 5 | 2.5 | 3.1 |
| 6 | 3.6 | 9.8 |
| 7 | 4.7 | 5.2 |
| 8 | 4.4 | 7.1 |
| 9 | 3.1 | 5.8 |
| 10 | 4.0 | 6.9 |
| Average | 4.7 | 7.4 |

6.0

### Example 2 (GeneChip Analysis Using Mesenchymal Stem Cells)

GeneChip analysis using mesenchymal stem cells was carried out to clarify the function of 1,5-AG in a human. After 1,5-AG was added to a medium to ensure that the final concentration of 1,5-AG became not more than 1 μg/mL or 40 μg/mL and the mesenchymal stem cells were cultured for 10 days, total RNA was collected to carry out GeneChip analysis so as to analyze genes which were expressed and fluctuated according to the difference in the amount of 1,5-AG cyclopaedically. A similar test was carried out 3 times to obtain results which were then summarized and analyzed. The test was carried out by the following method.

Material to be Tested

Crystalline 1,5-AG was dissolved in pure water to a concentration of 40 mg/mL and filtered with a centrifugal ultrafiltration membrane having a molecular weight cut-off of 3,000 to remove trace macromolecules.

The same pure water as water used to dissolve 1,5-AG was filtered with a centrifugal ultrafiltration membrane having a molecular weight cut-off of 3,000 to prepare a sample.

Cells

Mesenchymal cells derived from bone marrow (Riken Cell Bank, Resource No. MSC-R46)

Reagent

Dulbecco's Modified Eagle's Medium (DEMEM), 0.25% Trypsin/EDTA solution, RNeasy Plus Micro Kit (QIAGEN, Cat. No. 74034), QIAshredder (QIAGEN, Cat. No. 79654)

Medium

DMEM prepared by adding 10% fetal bovine serum (FBS) and a 3 ng/mL fibroblast growth factor (FGF-2) (to be referred to as “growth medium” hereinafter) was used for the preculture of the mesenchymal stem cells. 10% FBS-added DMEM (to be referred to as “test medium” hereinafter) was used in this test. FGF-2 has the function of keeping the undifferentiated state of the mesenchymal stem cells, and the undifferentiated state of the cells was maintained by using the FGF-2-added medium during preculture. In this test, to analyze the differentiation-inducing effect of the sample, a medium containing no FGF-2 was used.

Preculture of Human Mesenchymal Stem Cells

The human mesenchymal cells were initiated in a T-75 flask by using the growth medium and cultured in a CO₂ incubator (5% CO₂, 37° C., moistening). Medium exchange was carried out every 2 days, and the cells were separated by using 0.25% Trypsin/EDTA when 80% confluence was reached and collected to be used in this test.

Preparation of Sample

The sample was added to a test medium to ensure that the final concentration of 1,5-AG became 40 μg/mL. Stated more specifically, a 40 mg/mL 1,5-AG aqueous solution was added to the test medium in a ratio of 1/1,000 to ensure that the final concentration became 40 μg/mL and sterilized with a filter. As a comparative sample, water treated under the same procedure as the 1,5-AG aqueous solution was added to a test medium in a ratio of 1/1,000 and sterilized with a filter.

Cell Culture and Sample Treatment in this Test

A test medium was used to adjust the mesenchymal stem cells to a concentration of 18,000 cells/1 mL/well, and the cells were scattered on a 12-well plate to be cultured in a CO₂ incubator (5% CO₂, 37° C.) for 24 hours. After culture, the medium was replaced by a 1,5-AG added test medium and a comparative test medium for culture in a CO₂ incubator. On 4^{th} day after the addition of the sample, these mediums were replaced by a new 1,5-AG-added medium and a new comparative medium (1 mL) every 3 days to continue culture for 22 days at longest. On the first day after the addition of the sample and every 3 days after that, the form of each cell was observed through a phase difference microscope. On the seventh, tenth and thirteenth days after the addition of the sample, total RNA was extracted. These procedures were repeated three times each.

Extraction of Total RNA

The collection of total RNA from the cells was carried out by using the RNeasy plus mini kit.

GeneChip Analysis

GenecChip analysis was carried out by using the GeneChip Human Genome U133 Plus 2.0 Array of Affymetrix Co. As total RNA, an RNA having the highest purity and high yield was used out of RNA's extracted from the cells on 10^{th} day after the addition of the sample to carry out GeneChip analysis in a 1,5-AG added section and a comparative section once.

### (Results)

When the content of 1,5-AG in the medium of mesenchymal embryo cells to which 40 μg/m 1,5-AG was added and the content of 1,5-AG in a medium to which water was added were measured, the content in the 1,5-AG-added medium was 39 μg/mL and the content in the not-added medium was not more than 1 μg/mL. 39 μg/mL is almost equal to the high normal concentration of 1,5-AG in the human blood, thereby making it possible to establish a human cell culture model system having a normal 1,5-AG value.

As a result of GeneChip analysis, when the content of 1,5-AG was reduced from 40 μg/mL to 1 μg/mL or less, interleukin 6 signal transducer (IL6ST) gene was observed as a gene whose expression was reduced most. Then, 6 genes having unknown functions followed this and a gene for coding pregnancy specific beta-1-glycoprotein 1 (PSG-1 gene) was observed after these. IL6ST is the receptor of cytokine and the receptor of a ciliary neurotrophic factor which is connected with the growth of nerves. The protein coded by PSG-1 gene is a pregnancy specific protein which is secreted from mother's placenta in large quantities in pregnancy. It is said that when the secretion level of this protein is low in pregnancy, the risk of fetal malformation becomes high. The structure of this protein belongs to an immunoglobulin super family. It is reported that this family serves for cell attachment to a nervous system or immune system, or promotes neuritogenesis. Although PSG-1 is a gene related to pregnancy, the expression of CSH-1 decreases as well.

### Example 3 (Expression of PSG-1 and CSH-1 in Placental Trophoblastic Cells)

The influence upon these two genes (PSG-1 and CSH-1) of 1,5-AG was analyzed by a real-time RT-PCR method using placental trophoblastic cells. As a preliminary study, the expression level of PSG-1 was analyzed at three concentrations (1 μg/mL or less, 40 μg/mL and 160 μg/mL of 1,5-AG) and for three sampling times (3, 24 and 72 hours). A test was conducted by the following method.

Cells

Human placental villous trophoblastic cells (ScienCell Research Laboratories)

Medium

Trophoblast Medium (ScienCell Research Laboratories)

25 mL of fetal bovine serum (FBS) attached to the medium and 5 mL of a trophoblast growth supplement and 5 mL of a penicillin/streptomycin solution were added to 500 mL of a base medium.

Reagent

0.25% trypsin/EDTA solution (Nacalai tesque, Cat. No. 32777-44), Dulbecco's PBS (Nissui Pharmaceutical Co., Ltd., Cat. No. 05913), FastLane Cell cDNA for use in real-time RT-PCR (QIAGEN, Cat. No. 215011), SYBER Premix Ex Taq (Takara, Cat. No. RR041L), Oligonucleotides (primers) (FASMAC)

Method

Passage culture of human placental villous trophoblastic cells

The human placental villous trophoblastic cells were initiated in a T-75 flask by using a trophoblast medium (to be simply referred to as “medium” hereinafter) to be cultured in a CO₂ incubator (5% CO₂, 37° C., moistening). Medium exchange was carried out every 2 days, the cells were collected when 80% confluence was reached, and cells having a passage number of 2 were used in this test. Stated more specifically, after the cells were washed with D-PBS (−), the cells were separated by using 0.25% trypsin-EDTA, and a medium was added to neutralize trypsin. Then, the supernatant was removed by centrifugation (180 g, 5 minutes), a new medium was added to stir the cells, the number of the cells was counted by using a corpuscle calculating board, and a medium was used to achieve a target concentration (4.0×10³ cells/100 μL).

Preparation of Sample

The sample was added to mediums to achieve 1,5-AG final concentrations of 40 μg/mL and 160 μg/mL. When the final concentration was not more than 1 μg/mL, 1,5-AG was not added. Stated more specifically, a 1,5-AG aqueous solution having a concentration of 40 mg/mL was added to a test medium in a ratio of 1/1000 or 1/250 to achieve a final concentration of 40 μg/mL or 160 μg/mL, and the 1,5-AG solution was sterilized with a filter to be used in the test. In a test group, water treated under the same conditions as the 1,5-AG aqueous solution was added to a test medium in a ratio of 1/1000 and sterilized with a filter to be used in the test.

Cell Culture and Sample Treatment in this Test

A medium was used to adjust the cells to a concentration of 4×10³ cells/100 μL/well, and the cells were scattered on a 96-well plate to be cultured in a CO₂ incubator (5% CO₂, 37° C., moistening) for 24 hours. After culture, the medium was replaced by a 1,5-AG-containing medium and a comparative medium for culture in a CO₂ incubator. After the addition of the sample, the cells were cultured for 3 hours, 24 hours and 72 hours, the form of each cell was observed through a phase difference microscope, and total RNA was extracted. These procedures were repeated four times each. For reference, the 1,5-AG-containing medium and the comparative medium were exchanged every 3 days, and the form of each cell which was cultured for 10 days at longest was observed.

Extraction of Total RNA and Synthesis of cDNA

The collection of total RNA from the cells and the synthesis of cDNA were carried out according to the procedure of the FastLane Cell RT-PCR kit.

Real-Time PCR

Real-time PCR was carried out by an intercalator method using SYBR Green I. Stated more specifically, a reaction solution having composition shown in Table-2 below was prepared on a plate special for real-time PCR to carry out a PCR reaction.

TABLE 2

| Reagent | Amount |
|---|---|
| dsH₂O | 3.0 μL |
| SYBR Premix Ex Taq | 5.0 μL |
| Forward primer (5 μM) | 0.75 μL |
| Reverse primer (5 μM) | 0.75 μL |
| Synthesized cDNA | 0.5 μL |
| Total volume | 10.0 μL |

The PCR reaction was carried out under the conditions of 95° C.·30 sec−(95° C.·10 sec−60° C.·30 sec)×45 cycles−95° C.·15 sec−55° C.·15 sec−95° C.·15 sec, and the dissociation curve of the PCR product was obtained after the end of the PCR reaction. The relative expression level was calculated by the [symbol] Ct method to ensure that the expression level of a control became 1.0. Glyceraldehyde 3-phosphate dehydrogenase (GAPDH) gene was used as an internal standard gene.

Analysis of PSG-1 Gene Expression

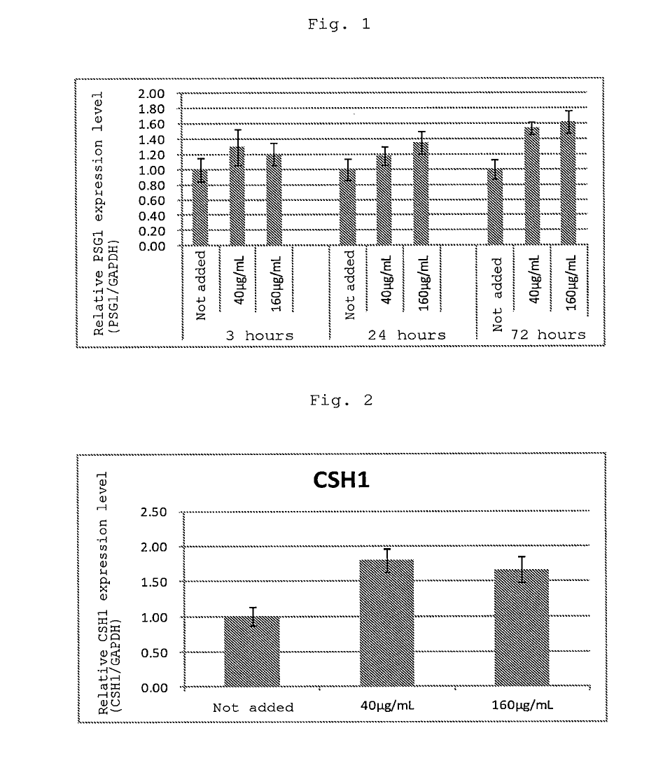

The results of the analysis of PSG-1 gene expression are shown in FIG. 1. In 1,5-AG added sections, the expression level of PSG-1 gene rose in proportion to the sample treatment time. After 72 hours of the sample treatment, the expression level in a 40 μg/mL 1,5-AG added section became 1.54 times that of a non-added section, and the expression level in a 160 μg/mL 1,5-AG added section became 1.62 times that of the non-added section. Thus, a significant difference between the added section and the non-added section was detected (P<0.05). Therefore, it was demonstrated that 1,5-AG has the effect of promoting the expression of PSG-1 gene in placental trophoblastic cells.

There was no big difference in the expression level of PSG-1 between the 40 μg/mL 1,5-AG added section and the 160 μg/mL 1,5-AG added section. Therefore, it is considered that the existence of 40 μg/mL 1,5-AG is enough to promote the expression of PSG-1 gene in this test system. It is estimated that a human generally takes about 5 to 10 mg of 1,5-AG from food per day (Nonpatent Document, clinical research and clinical examination into changes of 1,5-anhydroglucitol value in blood 38:485-488, 1994). There is also 1,5-AG biosynthesized in vivo. The content of 1,5-AG in the blood is maintained at about 20 to 40 μg/mL. However, when 1,5-AG is ingested temporarily in an amount of, for example, 20 g which is about 1,000 times the amount taken from food, the 1,5-AG level in the blood becomes high temporarily but returns to the original value in about 24 hours as 1,5-AG is eliminated through urine. It is considered from this result that the content of 1,5-AG required in the blood is about 20 to 40 μg/mL and excess 1,5-AG is excreted as it is not necessary. Since there is no change in the expression level of PSG-1 when the content of 1,5-AG is not less than 40 μg/mL according to data on placental cells, 40 μg/mL 1,5-AG suffices and when there is a shortage, it affects the expression of PSG-1.

The analysis of CSH-1 gene expression was carried out at the same time by using RNA extracted from the cells after 72 hours of the sample treatment.

Analysis of CSH-1 Gene Expression

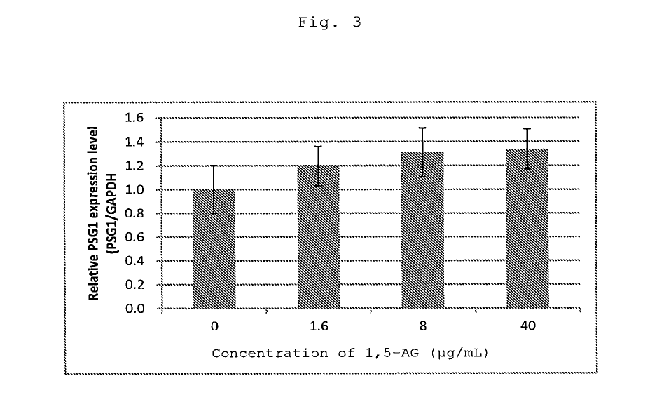

The results of the analysis of CSH-1 gene expression are shown in FIG. 2. The expression level of CSH-1 gene in a 1,5-AG non-added section was 1/1.80 times that in a 40 μg/mL 1,5-AG added section, and the expression level of CSH-1 gene in a 160 μg/mL 1,5-AG added section was 0.92 time that in a non-added section. Thus, the expression of CSH-1 gene was significantly suppressed in the non-added section as compared with the 40 μg/mL 1,5-AG added section (P<0.05). Therefore, it was demonstrated that 1,5-AG has the effect of promoting the expression of CSH-1 gene in the placental trophoblastic cells. CSH-1 gene is expressed in the placental trophoblast cells, codes growth hormone-like peptide, promotes the supply of nutrition to a fetus by adjusting maternal metabolism, and controls the neoangiogenesis of a fetus.

### Example 4 (Expression of PSG-1 in Intestinal Tract Cells)

PSG-1 is a gene which is expressed in placental cells in large quantities in pregnancy. The influence of 1,5-AG upon the expression of PSG-1 in intestinal tract cells which is not connected with pregnancy was investigated.

Preparation of Sample

A test was carried out at five concentrations (1.6, 8, 40, 200 and 1,000 μg/mL) of 1,5-AG. Stated more specifically, a 1,5-AG aqueous solution having a concentration of 100 mg/mL was diluted with water to prepare 100 times-concentrated solutions having concentrations of 0.16, 0.8, 4, 20 and 100 mg/mL. These 100 times-concentrated solutions were each added to a medium in a ratio of 1/100 to achieve final concentrations of 1.6, 8 and 40 μg/mL and sterilized with a filter to be used in the test. As a negative control, water was added to a medium in a ratio of 1/100 and sterilized with a filter to be used in the test.

Cell Culture and Sample Treatment

A medium was used to adjust Caco-2 cells to a concentration of 1×10⁵ cells/0.3 mL/well, and the cells were scattered on a 48-well plate to be cultured in a CO₂ incubator (5% CO₂, 37° C.) for 14 days (medium was exchanged every other day). After culture, it was confirmed that the cells were differentiated into intestinal epidermoid cells, the medium was replaced by a sample-containing test medium and a control-containing test medium to culture the cells in a CO₂ incubator for 3 days, and total RNA was extracted from the cells to analyze the expression level of PSG-1 gene. These procedures were repeated five times each.

Extraction of Total RNA

Total RNA was extracted by using the FastLane Cell cDNA kit. Stated more specifically, after culture, the medium was removed, and buffer FCW (150 μL) attached to the FastLane Cell cDNA kit was added to each well and removed right away to wash the cells. Then, buffer FCP (60 μL) attached to the FastLane Cell cDNA kit was added to each well and incubated at room temperature for 5 minutes to elute total RNA and collect it in a 96-well plate. Total RNA was kept at −80° C.

Synthesis of cDNA

The synthesis of cDNA was carried out by using the QuantiTect reverse transcription kit. Stated more specifically, the extracted total RNA (2 μL) was mixed with 2 μL of gDNA wipeout buffer and 10 μL of RNase free water and incubated at 42° C. for 5 minutes, the reaction solution was transferred on ice, and 6 μL of a reverse transcription reaction master mix (1 μL of Quantiscript Reverse Transcriptase, 4 μL of Quantiscript RT buffer and 1 μL of RT Primer Mix) was added to this reaction solution and incubated at 42° C. for 30 minutes to carry out reverse transcription. Then, the reaction solution was treated at 95° C. for 3 minutes to deactivate the reverse transcriptase. This reaction solution was used as synthesized cDNA for real-time PCR. It was kept at −20° C. until it was used for analysis.

Real-Time RT-PCR

Real-time PCT was carried out by an intercalator method using SYBR Green I. Stated more specifically, a total of an 8 μL system consisting of 1 μL of synthesized cDNA, 2.6 μL of dsH₂O, 4 μL of SYBR Premix ExTag, 0.2 μL of Forward primer (5 μM) and 0.2 μL of Reverse primer (5 μM) was used. A PCR reaction was carried out under the conditions of 95° C.·30 sec−(95° C.·10 sec−60° C.·30 sec)×40 cycles−95° C.·15 sec−55° C.·15 sec−95° C.·15 sec. The relative expression level was calculated by the [symbol] Ct method. GAPDH gene was used as an internal standard gene. The following sequences were used as the primer.

PSG1-for: 5′-AGCTGCCCATCCCCTACAT-3′
PSG1-rev: 5′-GGCTCTGACCGTTTAGCCA-3′
GAPDH-for: 5′-CATCCCTGCCTCTACTGGCGCTGCC-3′
GAPDH-rev: 5′-CCAGGATGCCCTTGAGGGGGCCCTC-3′

PSG-1 Production Promotion Test

Total RNA was extracted from the cells to carry out the analysis of PSG-1 gene expression by the real-time RT-PCT method. The results of gene expression analysis are shown in FIG. 3. It was observed that the average expression level of PSG-1 gene tends to become higher in an 1,5-AG added section than that in a non-added section. The expression level of PSG-1 gene in a 8 μg/mL test section was 1.31 times that in a non-added section and the expression level of PSG-1 in a 40 μg/mL test section was 1.34 times that in the non-added section.

### Example 5 (Effect of Promoting Neuritogenesis)

The promotion of the expression of a receptor related to neurogenesis was observed in a test on mesenchymal embryo cells. PSG-1 belongs to an immunoglobulin super family which includes some having the function of promoting neurogenesis. Then, the influence of 1,5-AG upon neuritogenesis was investigated. This was evaluated in a test system using PC12 cells which are generally used for the evaluation of neuritogenesis.

Cells

PC12 cells (Lot No. 48, Lot No. 51) (RIKEN cell bank, material 1)

Medium

### Growth Medium

DMEM medium prepared by adding 10% of FBS, 10% of HS and 1% of penicillin-streptomycin

### Test Medium

DMEM medium prepared by adding 0.1% of FBS, 0.1% of HS, 1 ng/mL NGF and 1% of penicillin-streptomycin

Reagent

DMEM medium (nacalai tesque, Cat. No. 08456-65), fetal bovine serum (FBS) (Cell Culture Bioscience, Cat. No. 171012), horse serum (HS) (Gibco, Cat. No. 16050), Penicillin-streptomycin solution (Nacalai tesque, Cat. No. 26253-84), 0.05% Trypsin-EDTA (GIBCO, Cat. No. 25300-062), Dulbecco's PBS (Nissui Pharmaceutical Co., Ltd., Cat. No. 05913), DPBS Calcium Magnesium (Gibco, Ca. No. 14040-133), live cell number measuring reagent SF (nacalai tesque, Cat. No. 25300), Neurite Outgrowth Staining Kit (Lifetechnolgoies, Cat. No. A15001)

Method

### Passage Culture of PC12

PC12 cells were initiated in a T-75 flask by using a growth medium to be cultured in a CO₂ incubator (5% CO₂, 37° C., moistening). Medium exchange was carried out every 2 days, the cells were collected when 80% confluence was reached, and cells having a passage number of 2 were used in this test. Stated more specifically, after the cells were washed with D-PBS (−), the cells were separated by using 0.05% trypsin, and a growth medium was added to neutralize trypsin. Then, the supernatant was removed by centrifugation (180 g, 5 minutes), a growth medium was added to stir the cells, the number of the cells was counted by using a corpuscle calculating board, and a growth medium was used to achieve a target concentration (2.0×10³ cells/100 μL).

Preparation of Sample

A test was carried out at five concentrations (1.6, 8, 40, 200 and 1,000 μg/mL) of 1,5-AG. Stated more specifically, a 1,5-AG aqueous solution having a concentration of 100 mg/mL was diluted with water to prepare 100 times-concentrated solutions having concentrations of 0.16, 0.8, 4, 20 and 100 mg/mL. These 100 times-concentrated solutions were each added to a test medium in a ratio of 1/100 to achieve final concentrations of 1.6, 8, 40, 200 and 1,000 μg/mL and sterilized with a filter to be used in the test. As a negative control, water was added to a medium in a ratio of 1/100 and sterilized with a filter to be used in the test. As a positive control, a test medium containing a 100 ng/mL nerve growth factor (NGF) was used.

Cell Culture and Sample Treatment in this Test

A growth medium was used to adjust the cells to a concentration of 2.0×10³ cells/100 μL/well, and the cells were scattered in a collagen-coated 96-well plate to be cultured in a CO₂ incubator (5% CO₂, 37° C., moistening) for 24 hours. After culture, the medium was replaced by a sample-containing test medium (100 μL), and the cells were cultured in a CO₂ incubator for 96 hours. After the end of culture, an image of the cells in each well was taken with a phase difference microscope and used for the measurement of nuetrite outgrowth rate, and nuclei and cytoplasms were fluorescent dyed by using a neurite outgrowth staining kit to measure the number of living cells and the amount of neurite outgrowth. These procedures were carried out five times each.

Determination of the Number of Living Cells and the Amount of Neurite Outgrowth Using a Neurite Outgrowth Staining Kit

After the end of culture, the number of living cells and the amount of neurite outgrowth were determined by using a neurite outgrowth staining kit. Stated more specifically, after the medium was removed, 100 μL of a staining solution (prepared by diluting Cell Viability Indicator and Cell Membrane Stain attached to the kit with calcium magnesium-containing PBS 1,000 times) was added to each well to incubate the cells at 37° C. for 20 minutes. After incubation, the staining solution was removed, 100 μL of a background suppression solution (prepared by diluting the background suppression dye attached to the kit with calcium magnesium-containing PBS 100 times) was added to each well so as to measure fluorescence intensity with a micro plate reader (measurement of the number of living cells: excitation wavelength of 495 nm, fluorescence wavelength of 515 nm, measurement of the amount of neurite outgrowth: excitation wavelength of 555 nm, fluorescent wavelength of 565 nm).

Determination of Neurite Outgrowth Rate

Based on the image taken above, the total number of cells, the number of cells showing the same or higher neurite outgrowth than the cell length and the number of cells showing neurites having a length 2 times or more than the cell length were measured from the image of the cells. The percentage (%) of cells showing neurite outgrowth was calculated from the measurement values.

Determination of Neurite Outgrowth Rate

First, in an NGF added section, cells showing an neurite outgrowth rate of 1 time or more accounted for 68.8%, and cells showing a neurite outgrowth rate of 2 times or more accounted for 54.5%. Thus, the distinct effect of promoting neurite outgrowth was observed. Then, as for 1,5-AG added sections, cells showing a neurite outgrowth rate of 1 time or more accounted for 12.6% of all the cells in a non-added section whereas cells showing a neurite outgrowth rate of 1 time or more accounted for 15.8% in a 40 μg/mL 1,5-AG added test section and 18.7% in a 200 μg/mL 1,5-AG added test section. Cells showing a neurite outgrowth rate of 2 times or more accounted for 5.7% in the non-added section whereas cells showing a neurite outgrowth rate of 2 times or more accounted for 10.3% and 10.4% in the above 1,5-AG added test sections, respectively, i.e., about 2 times that in the non-added section. Therefore, it was found that 1,5-AG is involved in the promotion of neuritogenesis.

Fluorescence Determination

Since the number of living cells in all test sections was about the same as that of a negative control (non-added) section, it was considered that 1,5-AG does not have an influence on the proliferation of PC12 cells. As for the amount of neurite outgrowth per cell, a significant increase of 183.1% was observed in an NGF added section as compared with the non-added section. Next, as for the 1,5-AG added sections, the amount of neurite outgrowth was 113.4% in a 8 μg/mL section and 113.3% in a 40 μg/L section as compared with the non-added section.

As a result of this test, the neurite outgrowth rate and the amount of outgrowth increased in 1,5-AG added sections, mainly a 40 μg/mL section.

### Example 6

Crystalline 1,5-AG was ground with a mortar and then mixed with commercially available modified milk powders to obtain 1,5-AG-containing modified milk powders. When the contents of 1,5-AG in the obtained milk powders were measured, they were 7.7, 38.5, 77, 154, 770 and 3,850 μg/g.

### EFFECT OF THE INVENTION

According to the present invention, there is provided a modified milk powder which enables babies who are fed with powder milk or both powder milk and breast milk to take 1,5-AG from powder milk like breast milk. 1,5-AG which is required for fetuses and infants can be supplied by adding 1,5-AG to powder milk for pregnant women and nursing mothers. The modified milk powder of the present invention can be taken in the same manner as when breast milk containing 1,5-AG which has various useful functions in vivo, for example, neurogenesis and angiogenesis is taken.

1. A modified milk powder containing 1,5-anhydro-D-glucitol in an amount of 7.7 to 3,850 μg/g.

2. The modified milk powder according to claim 1, wherein 1,5-anhydro-D-glucitol is obtained by the microbial reduction of 1,5-anhydro-D-fructose.


SEQUENCE LISTING (4 sequences)

SEQ ID NO: 1 - DNA, 19 bp, Artificial Sequence
  Forward primer PSG-1
agctgcccatcccctacat

SEQ ID NO: 2 - DNA, 19 bp, Artificial Sequence
  Reverse primer PSG-1
ggctctgaccgtttagcca

SEQ ID NO: 3 - DNA, 25 bp, Artificial Sequence
  Forward primer GAPDH
catccctgcctctactggcgctgcc

SEQ ID NO: 4 - DNA, 25 bp, Artificial Sequence
  Reverse primer GAPDH
ccaggatgcccttgagggggccctc